(19) United States
(12) Patent Application Publication
(10) Pub. No.: US 2017/0175182 A1
(43) Pub. Date: Jun. 22, 2017

(54) TRANSPOSASE-MEDIATED BARCODING OF FRAGMENTED DNA

(71) Applicant: AGILENT TECHNOLOGIES, INC., Santa Clara, CA (US)

(72) Inventors: Brian Jon Peter, Los Altos, CA (US); Robert A. Ach, San Francisco, CA (US); Alicia Scheffer-Wong, San Jose, CA (US)

(21) Appl. No.: 15/290,622

(22) Filed: Oct. 11, 2016

Related U.S. Application Data
(60) Provisional application No. 62/269,716, filed on Dec. 18, 2015.

(51) Int. Cl. C12Q 1/68

(52) U.S. Cl. CPC C12Q 1/6874

(57) ABSTRACT

Provided herein, among other things, are a variety of methods that comprise inserting a plurality of barcoded transposons into a population of DNA fragments that comprise DNA fragments of less than 1 kb in length, to produce transposon-tagged fragments that each comprise a barcoded transposon. Kits for performing this method are also provided.

### CROSS-REFERENCING

This application claims the benefit of provisional application Ser. No. 62/269,716, filed on Dec. 18, 2015, which application is incorporated by reference herein.

### BACKGROUND

Next-generation Sequencing (NGS) technologies have made whole-genome sequencing (WGS) routine, and various target enrichment methods have enabled researchers to focus sequencing power on the most important regions of interest. However, there is still a need for better methods of applying NGS to difficult target DNAs, such as formalin-fixed, paraffin-embedded (FFPE) solid tumor samples or cell-free or circulating tumor DNA (cfDNA/ctDNA.) Two of the biggest problems in sequencing these targets are that the amounts of DNA can be very small, and the allele frequencies can be very low, necessitating the capture of many DNA templates.

In particular, it can be challenging to sequence smaller fragments of genomic DNA using transposon-based methods because many of those methods require the insertion of two adjacent transposons produce a fragment. Small fragments are often not long enough to accommodate adjacent transposons and, as such, such transposon-based methods do not result in capture of the “ends” of small fragments.

### SUMMARY

Provided herein, among other things, is a method comprising: (a) inserting a plurality of barcoded transposons into a population of DNA fragments that comprise DNA fragments of less than 1 kb in length, to produce transposon-tagged fragments that each comprise a barcoded transposon, (b) adding an oligo-dN tail to both ends of the transposon-tagged fragments; (c) amplifying, by PCR, a population of 5′ products each comprising a barcode using i. a reverse primer that hybridizes to the top strand of the transposon and ii. a primer that hybridizes to the oligo-dN tail; and (d) amplifying, by PCR, a population of 3′ products each comprising a barcode using i. a forward primer that hybridizes to the bottom strand of the transposon and ii. the primer that hybridizes to the oligo-dN tail. After the products are sequenced, the 5′ and 3′ sequences can be assembled by matching sequence reads that have the same barcode. A kit for performing this method is also provided.

Some embodiments of this method may offer certain advantages depending on how the method is performed. For example, some other methods of barcoding DNA fragments require two ligation events per strand to capture a fragment. As such, in order to capture both the top and bottom strands, eight ends must be ligated to produce four bonds. If either the 5′ or 3′ end of a fragment has any chemical damage, that strand will necessarily be unligatable and lost. Notably, various DNA repair kits can repair 3′ blocked ends but not 5′ blocked ends. In contrast, the present method requires only one transposition event into the middle of the DNA, thereby barcoding both strands and, even if one or both 5′ ends of the fragment are blocked, that sequence can still be recovered.

Further, for certain applications such as when the method is applied to analyze circulating tumor DNA or DNA recovered from an FFPE sections, the amount of DNA in the sample is limiting and, as such, there is a need to capture as many individual molecules as possible. Some current methods of library construction have efficiencies around 10%, meaning 10% of the DNA molecules are converted into adapter-ligated, sequenceable molecules. Ligation based methods require 5′ phosphorylated ends, which must be created by enzymatic steps after shearing. Thus, if the 5′ ends are damaged, or if the 5′ end is not efficiently phosphorylated, or if DNA ligase activity is inhibited in any way, the efficiency will be lower. Furthermore, may transposon-based library preparation methods require two insertion sites per target fragment, while the present method requires only a single insertion per target DNA, and only requires intact/repaired 3′ ends. Thus, target fragment can be captured at a higher efficiency using the present method.

Finally, library preparation methods that rely on ligation of sequencing adapters to one or both ends of the target DNA fragment are susceptible to creating dimers of the adapters, which must be removed or designed around. Removal of adapter dimers by solid phase reversible immobilization (SPRI) or gel purification is laborious and can reduce efficiency of DNA capture. The present method does not require ligation of adapters and, as such, there is no opportunity for adapter dimers to form.

Also provided herein, among other things, is a method comprising: inserting a plurality of barcoded transposons into a population of DNA fragments that comprise DNA fragments of less than 1 kb in length, to produce transposon-tagged fragments that each comprise a barcoded transposon; circularlizing the transposon-tagged fragments; and amplifying, by inverse PCR, a population of products each comprising a barcode and the sequence of a DNA fragment. A kit for performing this method is also provided.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an embodiment of the present method.

FIG. 2 schematically illustrates a method by which sequences can be assembled.

FIG. 3 schematically illustrates another embodiment of the first method.

FIG. 4 schematically illustrates the architecture of transposon that could be used in some embodiments of the method.

FIG. 5 schematically illustrates an embodiment of a second method.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, A., Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “sample” as used herein relates to a material or mixture of materials, typically, although not necessarily, in liquid form, containing one or more analytes of interest. In one embodiment, the term as used in its broadest sense, refers to any plant, animal or viral material containing DNA or RNA, such as, for example, tissue or fluid isolated from an individual (including without limitation plasma, serum, cerebrospinal fluid, lymph, tears, saliva and tissue sections), from preserved tissue (such as FFPE sections) or from in vitro cell culture constituents, as well as samples from the environment.

The term “nucleic acid sample,” as used herein denotes a sample containing nucleic acids. A nucleic acid samples used herein may be complex in that they contain multiple different molecules that contain sequences. Genomic DNA samples from a mammal (e.g., mouse or human) are types of complex samples. Complex samples may have more than 10⁴, 10⁵, 10⁶ or 10⁷ different nucleic acid molecules. Also, a complex sample may comprise only a few molecules, where the molecules collectively have more than 10⁴, 10⁵, 10⁶ or 10⁷ or more nucleotides. A DNA target may originate from any source such as genomic DNA, or an artificial DNA construct. Any sample containing nucleic acid, e.g., genomic DNA made from tissue culture cells or a sample of tissue, may be employed herein.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution and a number of different elements attached to a solid support at random positions (i.e., in no particular order). A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

The term “primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with their use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing and elongating steps are performed multiple times such that the amount of amplification product is increasing, often times exponentially, although exponential amplification is not required by the present methods. Amplification typically requires the presence of deoxyribonucleo side triphosphates, a DNA polymerase enzyme and an appropriate buffer and/or co-factors for optimal activity of the polymerase enzyme. The term “amplification product” refers to the nucleic acid sequences, which are produced from the amplifying process as defined herein.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

In certain cases, an oligonucleotide used in the method described herein may be designed using a reference genomic region, i.e., a genomic region of known nucleotide sequence, e.g., a chromosomal region whose sequence is deposited at NCBI's Genbank database or other database, for example. Such an oligonucleotide may be employed in an assay that uses a sample containing a test genome, where the test genome contains to binding sites for sequences in the oligonucleotide.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

If two nucleic acids are “complementary”, they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “covalently linking” refers to the production of a covalent linkage between two separate molecules, e.g., the top and bottom strands of a double stranded nucleic acid. Ligating is a type of covalent linking.

The term “genotyping”, as used herein, refers to any type of analysis of a nucleic acid sequence, and includes sequencing, polymorphism (SNP) analysis, and analysis to identify rearrangements.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, Life Technologies, Pacific Bio, and Roche etc. Next-generation sequencing methods may also include nanopore sequencing methods or electronic-detection based methods such as Ion Torrent technology commercialized by Life Technologies.

The term “extending”, as used herein, refers to the extension of a primer by the addition of nucleotides using a polymerase. If a primer that is annealed to a nucleic acid is extended, the nucleic acid acts as a template for extension reaction.

The term “barcode sequence” or “molecular barcode”, as used herein, refers to a unique sequence of nucleotides used to a) identify and/or track the source of a polynucleotide in a reaction and/or b) count how many times an initial molecule is sequenced (e.g., in cases where substantially every molecule in a sample is tagged with a different sequence, and then the sample is amplified). Barcode sequences may vary widely in size and composition; the following references provide guidance for selecting sets of barcode sequences appropriate for particular embodiments: Casbon (Nuc. Acids Res. 2011, 22 e81), Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular embodiments, a barcode sequence may have a length in range of from 4 to 36 nucleotides, or from 6 to 30 nucleotides, or from 8 to 20 nucleotides.

The term “barcoded transposon”, as used herein, refers to a transposon that contains a “degenerate base region” or “DBR” within its sequence, where the terms “degenerate base region” and “DBR” refers to a type of molecular barcode that has complexity that is sufficient to help one distinguish between fragments to which the DBR has been added. In some cases, substantially every tagged fragment may have a different DBR sequence. In these embodiments, a high complexity DBR may be used (e.g., one that is composed of at least 10,000 or 100,000 sequences). In other embodiments, some fragments may be tagged with the same DBR sequence, but those fragments can still be distinguished by the combination of i. the DBR sequence, ii. the sequence of the fragment, iii. the sequence of the ends of the fragment, and/or iv. the site of insertion of the DBR into the fragment. In some embodiments, at least 95%, e.g., at least 96%, at least 97%, at least 98%, at least 99% or at least at least 99.5% of the target polynucleotides become associated with a different DBR sequence. In some embodiments a DBR may comprise one or more (e.g., at least 2, at least 3, at least 4, at least 5, or 5 to 30 or more) nucleotides selected from R, Y, S, W, K, M, B, D, H, V, N (as defined by the IUPAC code).

A barcoded transposon can be made by making an oligonucleotide containing degenerate sequence (e.g., an oligonucleotide that has a run of 4-10 “Ns”) and then amplifying it, or a barcoded transposon can be made by making a number of oligonucleotides separately, mixing the oligonucleotides together, and by amplifying them en masse. In other words, the population of transposons used in the method can be made as a single oligonucleotide that contains degenerate positions (i.e., positions that contain more than one type of nucleotide). Alternatively, the population of transposons can be made by fabricating them individually or using an array of the oligonucleotides using in situ synthesis methods, cleaving the oligonucleotides from the substrate and then making them double stranded by PCR. Examples of such methods are described in, e.g., Cleary et al (Nature Methods 2004 1: 241-248) and LeProust et al (Nucleic Acids Research 2010 38: 2522-2540).

In some cases, a DBR may be error correcting. Descriptions of exemplary error identifying (or error correcting) sequences can be found throughout the literature (e.g., in are described in US patent application publications US2010/0323348 and US2009/0105959 both incorporated herein by reference). Error-correctable codes may be necessary for quantitating absolute numbers of molecules. Many reports in the literature use codes that were originally developed for error-correction of binary systems (Hamming codes, Reed Solomon codes etc.) or apply these to quaternary systems (e.g. quaternary Hamming codes; see Generalized DNA barcode design based on Hamming codes, Bystrykh 2012 PLoS One. 2012 7: e36852).

In some embodiments, a DBR may additionally be used to determine the number of initial target polynucleotide molecules that have been analyzed, i.e., to “count” the number of initial target polynucleotide molecules that have been analyzed. PCR amplification of molecules that have been tagged with a DBR results in multiple sub-populations of products that are clonally-related in that each of the different sub-populations is amplified from a single tagged molecule. As would be apparent, even though there may be several thousand or millions or more or molecules in any of the clonally-related sub-populations of PCR products and the number of target molecules in those clonally-related sub-populations may vary greatly, the number of molecules tagged in the first step of the method can be estimated by counting the number of DBR sequences associated with a target sequence that is represented in the population of PCR products. This number is useful because, in certain embodiments, the population of PCR products made using this method may be sequenced to produce a plurality of sequences. The number of different DBR sequences that are associated with the sequences of a target polynucleotide can be counted, and this number can be used (along with, e.g., the sequence of the fragment, the sequence of the ends of the fragment, and/or the site of insertion of the DBR into the fragment) to estimate the number of initial template nucleic acid molecules that have been sequenced.

The term “sample identifier sequence” is a type of barcode that can be appended to a target polynucleotide, where the sequence identifies the source of the target polynucleotide (i.e., the sample from which sample the target polynucleotide is derived). In use, each samples is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences.

The term “plurality of barcoded transposons”, as used herein, refers to a population of transposons that, collectively, contain multiple DBRs, were each transposon molecule contains one or two DBRs (which may be the same or different), and there are multiple barcodes represented in the population. There may be at least 10, at least 100, at least 1,000, at least 10,000, at least 100,000, at least 1M or at least 10M or more barcode sequences represented in a plurality of barcoded transposons in a population of barcoded transposons. In some embodiments, each transposon molecule contains a different barcode sequence, while in other embodiments, multiple transposon molecules may contain the same barcode sequence.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “top strand,” as used herein, refers to either strand of a nucleic acid but not both strands of a nucleic acid. When an oligonucleotide or a primer binds or anneals “only to a top strand,” it binds to only one strand but not the other. The term “bottom strand,” as used herein, refers to the strand that is complementary to the “top strand.” When an oligonucleotide binds or anneals “only to one strand,” it binds to only one strand, e.g., the first or second strand, but not the other strand.

The terms “reverse primer” and “forward primer” refer to primers that hybridize to different strands in a double-stranded DNA molecule, where extension of the primers by a polymerase is in a direction that is towards the other primer.

The term “a population of DNA fragments comprising DNA fragments of less than 1 kb in length”, as used herein, refers to a population of fragments in which at lest 1%, at least 5%, at least 10%, at least 20%, at least 30%, at least 50%, at least 80% or at least 90% of the fragment molecules are less than 1 kb in length. In some embodiments such a population may have a median fragment length in the range of 50-500 bp, e.g., 100-400 bp.

The term “transposon-tagged fragments”, as used herein, refers to a population of fragments into which barcoded transposons have been inserted. In some embodiments, the transposons are inserted at a density in the range of 1 every 50-800 bp, e.g., 60-200 bp, meaning that tagged fragment will contain, on average, a single transposon inserted into it.

The term “oligo-dN tail”, as used herein, refers to a tail of Gs, As, Ts or Cs added onto the 3′ end of a strand of a double stranded DNA molecule by the action of a terminal transferase. A tail may have 10 to more than 100 nucleotides.

The term “a primer that hybridizes to the oligo-dN tail”, as used herein, refers to a primer that has an oligo-dG, oligo-dA, oligo-dT or oligo-dC tract of 8-20 or more bases at its 3′ end, where those bases hybridize to a complementary homopolymeric tail added to a fragment.

The term “both ends of the transposon-tagged fragments”, as used herein, refers to both ends of a double stranded DNA molecule (i.e., the left hand end and the right hand end if the molecule is drawn out horizontally). If a terminal transferase is used to tail both ends of a population of fragments, the 3′ ends of one strand of a double stranded fragments as well as the 3′ end of the other strand of the fragments are tailed.

The terms “5′ sequence reads” and “3′ sequence reads”, as used herein, refer to sequence reads that correspond to opposing ends of a transposon tagged fragment (e.g., to the left of the transposon and to the right of the transposon, if the transposon tagged fragment is drawn out horizontally).

The term “the sequence of a barcode”, as used herein, refers to the sequence of nucleotides that makes up the barcode. The sequence of a barcode may be at lest 3 nucleotides in length, more usually 5-30 or more nucleotides in length.

The term “match”, as used herein, refers to an action in which two sequences are compared and if they are identical, complementary, or very similar (e.g., when error correcting barcodes are used) they are indicated as being a match. In some embodiments, matched sequences are placed into a group.

The term “assembling matched sequence reads”, as used herein, refers to a computationally implemented step in which matched reads (i.e., sequences that contain the same or very similar barcode or complement thereof) are aligned with one another to produce an assembled sequence that is made up of sub-sequences that are contributed by the matched sequences reads. In some embodiments, sequence assembly may involve making consensus sequences from sequence reads that have the same barcode, and then assembling a sequence from the consensus sequences.

The term “or variant thereof”, used herein, refers to a protein that has an amino acid sequence that at least 80%, at least 85%, at least 90%, at least 95%, at least 97%, at least 98% or at least 99% identical to a protein that has a known activity, wherein the variant has at least some of the same activities as the protein of known activity. For example, a variant of a wild type transposase should be able to catalyze the insertion of a corresponding transposon into DNA.

The term “circularlizing”, as used herein, refers to the intramolecular ligation of the ends of a molecular of DNA to one another, thereby producing a circle of DNA.

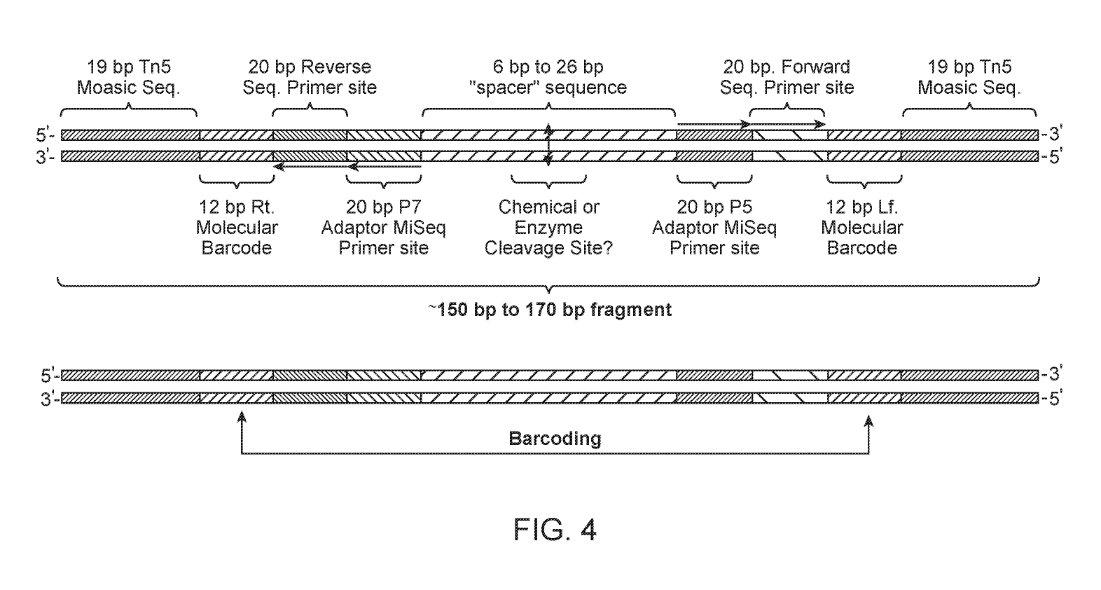

The term “inverse PCR”, as used herein, refers to a PCR reaction that uses a circular template in which the primers used point away from one another, as shown in FIG. 4.

The term “polishing”, as used herein, refers to an enzymatically-catalyzed reaction in which ragged ends (which may be a combination of 3′ overhangs, 5′ overhangs and blunt ends) blunt by the action of one or more polymerases (e.g., T4 DNA polymerase, etc.).

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed. As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

Provided herein are various ways for tagging samples containing DNA fragments that are of less than 1 kb in length. The methods can be employed to analyze genomic DNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the genomic DNA used in the method may be derived from a mammal, wherein in certain embodiments the mammal is a human. In exemplary embodiments, the sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In particular embodiments, a sample may be obtained from a subject, e.g., a human.

In some embodiments, the sample comprises DNA fragments obtained from a clinical sample, e.g., a patient that has or is suspected of having a disease or condition such as a cancer, inflammatory disease or pregnancy. In some embodiments, the sample may be made by extracting fragmented DNA from an archived patient sample, e.g., a formalin-fixed paraffin embedded tissue sample. In other embodiments, the patient sample may be a sample of cell-free circulating DNA from a bodily fluid, e.g., peripheral blood. The DNA fragments used in the initial steps of the method should be non-amplified DNA that has not been denatured beforehand. The sample may be fragmented mechanically (e.g., by sonication, nebulization, or shearing) or enzymatically, using a double stranded DNA fragmentase enzyme (New England Biolabs, Ipswich Mass.). In other embodiments, the DNA in the initial sample may already be fragmented (e.g., as is the case for FFPE samples and circulating cell-free DNA (cfDNA), e.g., ctDNA). The fragments in the initial sample may have a median size that is below 1 kb (e.g., in the range of 50 bp to 500 bp, 80 bp to 400 bp, or 100-1,000 bp), although fragments having a median size outside of this range may be used. Cell-free or circulating tumour DNA (ctDNA), i.e., tumour DNA circulating freely in the blood of a cancer patient, is highly fragmented, with a mean fragment size about 165-250 bp (Newman et al Nat Med. 2014 20: 548-54). cfDNA can be obtained by centrifuging whole blood to remove all cells, and then analyzing the remaining plasma.

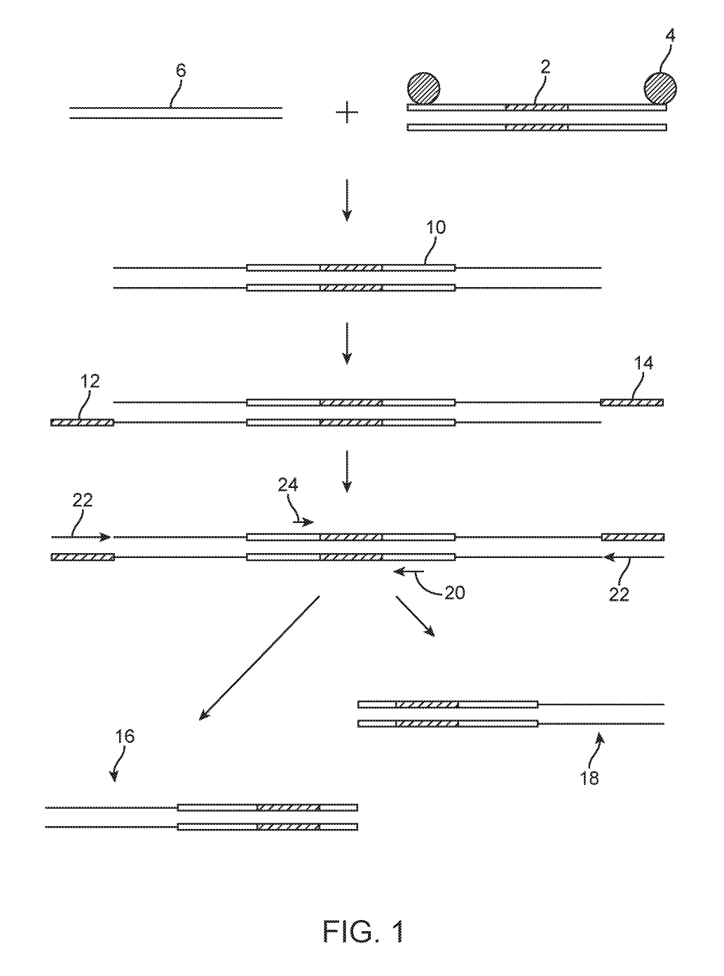

In some embodiments (and as shown in FIG. 1), the method may comprise: contacting a plurality of barcoded transposons 2 and a transposase 4 with a population of DNA fragments comprising DNA fragments of less than 1 kb in length 6, to produce transposon-tagged fragments 10 that each comprise a barcoded transposon. As shown, the transposase 4 is pre-loaded with barcoded transposons 2 to produce complexes which, as shown, may contain two transposase molecules per transposon molecule. The amount of transposase and transposon and/or sample used may be tailored to produce a transposon insertion every 80-500 bp (e.g., every 100-300 bp) so that a relatively high proportion of tagged fragments, e.g., at least 25%, at least 50%, or at least 75% of the tagged fragments, particularly smaller fragments that are less than 500 bp in length, receive, on average, a single insertion. The resulting population of tagged fragments 10 contains a population of fragments that contain a single barcoded transposon, where the complexity of the barcode in the population is sufficient to distinguish between overlapping or adjacent fragments from the same locus. In some embodiments, the complexity of the barcode in the population may be at least 12, at least 24, at least 96, at least 384, at least 10³, at least 10⁴ or at least 10⁵. This step of the method may be implemented in a similar way to the method described by U.S. Pat. No. 9,074,251 (Steemers) except that in the embodiment shown in FIG. 1 the transposon only contains a single barcode.

The transposase used in this step of the method may be a Tn transposase (e.g. Tn3, Tn5, Tn7, Tn10, Tn552, Tn903), a MuA transposase, a Vibhar transposase (e.g. from Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Te1, THE-1, Tn/O, TnA, Tn3, Tn5, Tn7, Tn10, Tn552, Tn903, Tol1, Tol2, Tn10, Ty1, including variants thereof. In some cases, the insertion can be facilitated and/or triggered by addition of one or more cations. The cations can be divalent cations such as, for example, Ca²⁺, Mg²⁺ and Mn²⁺.

Next, an oligo-dN tail (i.e., a homopolymer tail) may be added to both ends (i.e., to the 3′ ends of both strands) of the transposon-tagged fragments (i.e., adding tails 12 and 14) using a terminal transferase. In this step, it may be advantageous to add an appropriate ddNTP to the reaction (i.e., ddCTP, if the tail is a dC tail, etc.) in order limit the length of the tail. In these embodiments, the dNTP mix used may have both the dNTP and the corresponding ddNTP at a ratio in the range of 10:1 to 100:1, for example. The added tail may have an median length of 8-100 bases, e.g., 10-50 bases.

Next, the method may comprise i. amplifying, by PCR, a population of 5′ products 16 each comprising a barcode using i. a reverse primer 20 that hybridizes to the top strand of the transposon and ii. a primer 22 that hybridizes to the oligo-dN tail; and ii. amplifying, by PCR, a population of 3′ products 18 each comprising a barcode using i. a forward primer 24 that hybridizes to the bottom strand of the transposon and ii. the primer 22 that hybridizes to the oligo-dN tail. As would be apparent, because the forward and reverse primers point toward each other, these reactions may be done by performing an initial pre-amplification reaction using only primer 22 (which hybridizes to the oligo-dN tail), and then performing two separate reactions (one with primers 20 and 22 and the other with primers 24 and 22) after sufficient material has been amplified.

In certain embodiments, the method may further comprise sequencing at least some of 5′ products 16 and 3′ products 18. As would be apparent, in these embodiments, the transposon itself may contain sequences that are compatible with use in the sequencing platform being used for sequencing where those sequences are downstream from the primers. Alternatively, the primers used for amplification step may contain 5′ tails containing sequences that are compatible with use in the sequencing platform being used for sequencing or, the amplification products themselves may be amplified using primers that contain 5′ tails containing sequences that are compatible with use in the sequencing platform being used for sequencing. Alternatively, after tailing, an adaptor that has a 3′ end of 5-10 bases that is complementary to the tail and a 5′ tail that is compatible with a sequence platform may be ligated to the 5′ ends of both strands of the fragment, as described in Lazinski (BioTechniques 2013 54:25-34). The products may be sequenced using any suitable method including, but not limited to Illumina's reversible terminator method, Roche's pyrosequencing method (454), Life Technologies' sequencing by ligation (the SOLiD platform), Life Technologies' Ion Torrent platform or Pacific Biosciences' fluorescent base-cleavage method. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps.

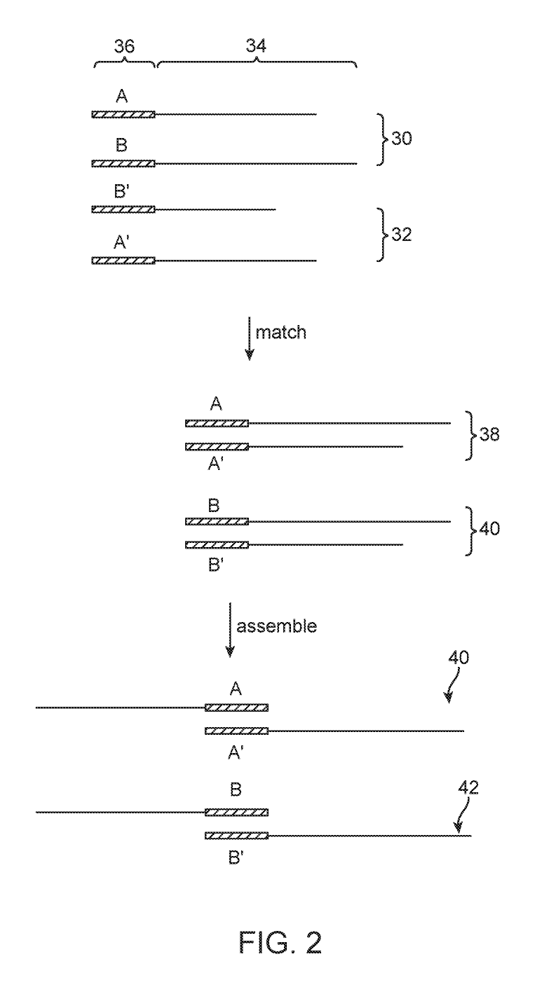

As shown in FIG. 2, the sequencing step results in a plurality of 5′ sequence reads 30 and a plurality of 3′ sequence reads 32, where the sequence reads each comprise i. the sequence of at least part of the sequence of a DNA fragment 34 and ii. the sequence of a barcode 36. Some of the 5′ sequence reads have the same barcode as the 3′ sequence reads (e.g., the same barcode sequence or the reverse complement of that sequence), and, as shown in FIG. 2, those sequence reads can be matched and assembled to obtain the entire sequence of fragments in the initial sample. As would be apparent, this part of the method may comprise comparing the barcodes in at least some of the sequence reads to one another to obtain matches 38 and 40, and assembling matched sequence reads to obtain the entire sequence of a DNA fragment of the initial sample (e.g., 42 and 44) The barcode may be at the 3′ end or the 5′ end of sequence read, depending on how which products are being sequenced and how the sequencing is done. As such, some sequences may need to be converted into their reverse complement prior to matching their barcodes or prior to sequence assembly. Further, the barcode sequences and other added sequences may be trimmed from the sequence reads prior to sequence assembly. Further, similar sequences that have the same barcode may be aligned with one another first to make a consensus sequence, thereby eliminating potential sequencing errors prior to assembly. FIG. 2 therefore schematically illustrates the general principle of these steps of the method.

As would be recognized, some of the analysis steps of the method, e.g., the comparing and assembly steps, can be implemented on a computer. In certain embodiments, a general-purpose computer can be configured to a functional arrangement for the methods and programs disclosed herein. The hardware architecture of such a computer is well known by a person skilled in the art, and can comprise hardware components including one or more processors (CPU), a random-access memory (RAM), a read-only memory (ROM), an internal or external data storage medium (e.g., hard disk drive). A computer system can also comprise one or more graphic boards for processing and outputting graphical information to display means. The above components can be suitably interconnected via a bus inside the computer. The computer can further comprise suitable interfaces for communicating with general-purpose external components such as a monitor, keyboard, mouse, network, etc. In some embodiments, the computer can be capable of parallel processing or can be part of a network configured for parallel or distributive computing to increase the processing power for the present methods and programs. In some embodiments, the program code read out from the storage medium can be written into memory provided in an expanded board inserted in the computer, or an expanded unit connected to the computer, and a CPU or the like provided in the expanded board or expanded unit can actually perform a part or all of the operations according to the instructions of the program code, so as to accomplish the functions described below. In other embodiments, the method can be performed using a cloud computing system. In these embodiments, the data files and the programming can be exported to a cloud computer that runs the program and returns an output to the user.

A system can, in certain embodiments, comprise a computer that includes: a) a central processing unit; b) a main non-volatile storage drive, which can include one or more hard drives, for storing software and data, where the storage drive is controlled by disk controller; c) a system memory, e.g., high speed random-access memory (RAM), for storing system control programs, data, and application programs, including programs and data loaded from non-volatile storage drive; system memory can also include read-only memory (ROM); d) a user interface, including one or more input or output devices, such as a mouse, a keypad, and a display; e) an optional network interface card for connecting to any wired or wireless communication network, e.g., a printer; and f) an internal bus for interconnecting the aforementioned elements of the system.

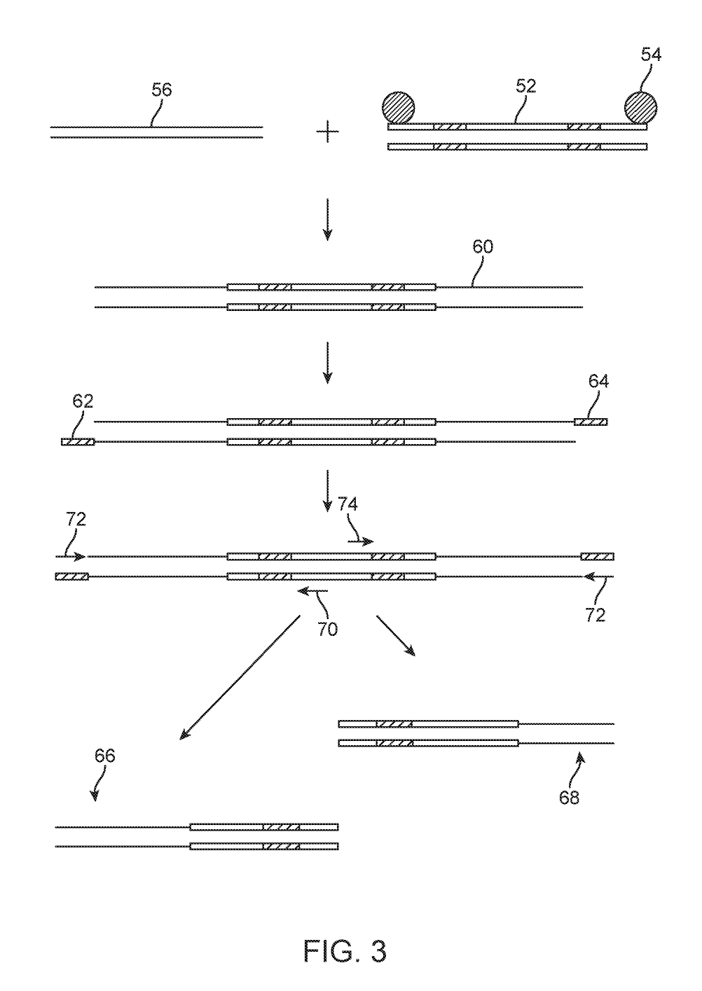

FIG. 3 shows a variation of the method illustrated in FIG. 1. This embodiment makes use of a barcoded transposon that contains two barcodes. The barcodes in the transposon can be the same or different. If the barcodes are different, then the barcodes in each transposon molecule can be linked via a look-up table or the like. In this embodiment, the method may comprise: (a) contacting a plurality of barcoded transposons 52 and a transposase 54 with a population of DNA fragments comprising DNA fragments of less than 1 kb in length 56, to produce transposon-tagged fragments 60 that each comprise a barcoded transposon. As shown, the transposon in this embodiment of the method has two barcodes. The remainder of this embodiment of the method is basically the same as that described above and as shown in FIG. 1 in that an oligo-dN tail is be added to both ends of the transposon-tagged fragments (tails 62 and 64) using a terminal transferase and then amplifying, by PCR, a population of 5′ products 66 each comprising a barcode using i. a reverse primer 70 that hybridizes to the top strand of the transposon and ii. a primer 72 that hybridizes to the oligo-dN tail; and amplifying, by PCR, a population of 3′ products 68 each comprising a barcode using i. a forward primer 74 that hybridizes to the bottom strand of the transposon and ii. the primer 62 that hybridizes to the oligo-dN tail, except that the forward and reverse primers are designed to not point toward each other. In these embodiment, the 3′ and 5′ products can, in theory, be amplified in a single PCR reaction. However, in some embodiments, an initial pre-amplification reaction using only primer 72 (which hybridizes to the oligo-dN tail) is performed, the sample is split, and then two separate reactions (one with primers 70 and 72 and the other with primers 74 and 72) are performed. The products of this method are essentially the same as the products produced in the method shown in FIG. 1, and they can be matched and assembled in the same as way as shown in FIG. 2.

One implementation of the method may be done by loading a purified transposase protein with a synthetically made double stranded transposon. Each end of the oligonucleotide contains the optimized transposase adapter sequence, bringing together two protein molecules into a single active nucleoprotein complex. Next, the transposase-DNA complex is incubated with the target DNA, under conditions where each 160-250 bp target of ctDNA receives, on average, a single transposase insertion. These conditions can be identified by titrating the ratio of target DNA fragments to active transposomes. Although longer DNAs provide more space for transposases to land in, Picelli et al. (Genome Research 2014 24:2033-2040) found that Tn5-based transposition could efficiently make libraries out of DNAs even down to 100 bp, even when each fragment needed two transposon landings. As the present method only requires a single transposition per fragment, it should be even more efficient for capturing short fragments. In the present method, the transposase-inserted sequence are designed such that insertion of the loop of DNA will add a molecular barcode into the middle of the fragments, together with optional sequences for sample indexing and amplification primer sites, and optionally, restriction enzyme sites or other cleavage sites. A schematic of some of these sites is shown in FIG. 4. This architecture could be used in the embodiments illustrated in FIG. 2 and FIG. 4. Note that the total length of the loop may need to be longer than 200 bp due to torsional constraints of duplex DNA. Longer duplex DNAs could be synthesized using synthetic biology methods. In some embodiments, a hybrid of ssDNA and dsDNA may be used; the ssDNA region would ensure connectivity while enabling enough flexibility for the transposase to bind both sites. After mixing and transposase insertion, the transposase may be inactivated by chemical addition, heat, or other means known in the art. In preferred embodiments, the transposase protein dissociates from, or is removed from, the target DNA, leaving only the inserted DNA. After the transposase is inactivated, the target DNA is incubated with terminal deoxynucleotidyl transferase (TdT) and a mixture of dNTPs and ddNTPs. This will create a homopolymer tail on the 3′ end of the fragments. This homopolymer tail can then be used as a primer binding site.

Next, the different parts of the target DNA sequence can be amplified using one set of primers against the homopolymer tails, and a second set of primers which bind in the inserted sequence. In some embodiments, the primers agains the homopolymer tails may have a 3′ nucleotide of a different sequence, ensuring that the primer aligns to the junction of the homopolymer and target sequence. For example, a target DNA with a polydA tail may be primed with a mixture of 5′(T20)dG, 5′(T20)dC, and 5′(T20)dA. The second set of primers should be positioned such that each primer is able to read through the molecular barcode; the single, unique molecular barcode will need to be used to associate the 5′ and 3′ ends of the target fragment. In some embodiments, there would be a chance that primers could create “primer dimer” short amplification products, if both the forward and reverse primers are added at the same time. However, this can be averted by first doing a few rounds of amplification using the homopolymer primers, and then splitting the amplification mix into two tubes: one contains the forward primer, and one contains the reverse primer, such that the same tube never contains both forward and reverse.

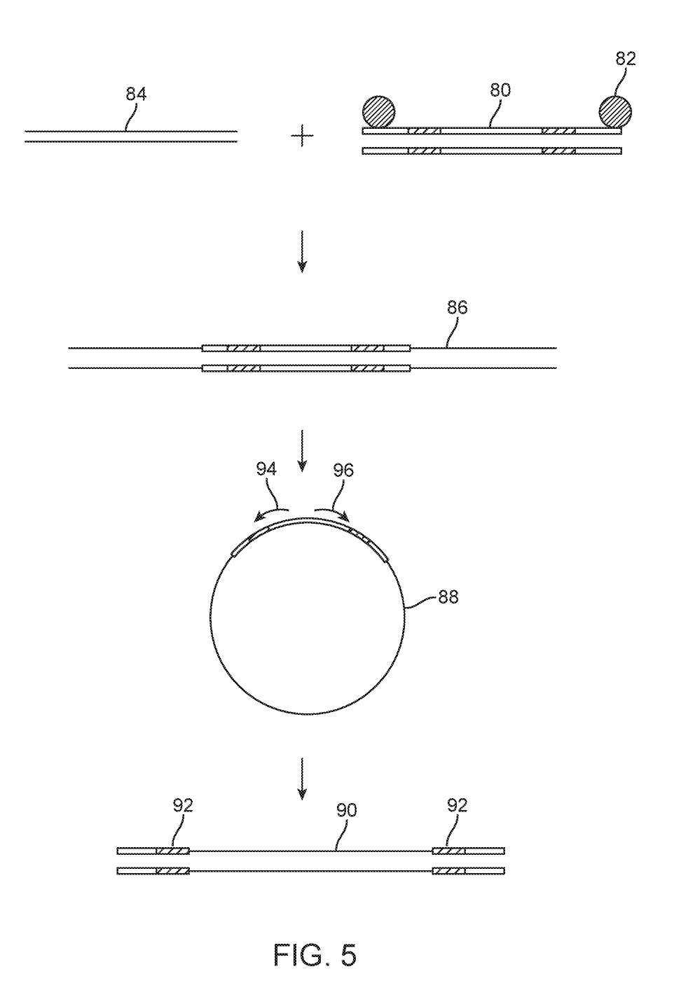

FIG. 5 shows an alternative method that comprises contacting a plurality of barcoded transposons 80 and a transposase 82 with a population of DNA fragments 84 comprising DNA fragments of less than 1 kb in length, to produce transposon-tagged fragments 86 that each comprise a barcoded transposon. This step of the method may be done as described above. In this method, the barcodes in the transposon can be the same or different and, if the barcodes are different, then the barcode sequences can be linked via a look-up table or the like. In this embodiment, the method may comprise repairing and polishing the ends, then circularlizing the transposon-tagged fragments (i.e., joining the ends of the transposon-tagged fragments together intramolecularly) to produce DNA circles 88; and then amplifying, by inverse PCR, a population of products 90. As shown, the amplification products comprise a barcode 90 and the sequence of a DNA fragment 92. The amplification step is done using i. a reverse primer 94 that hybridizes to the top strand of the transposon and ii. a forward primer 96 that hybridizes to the bottom strand of the transposon.

Next, the method may comprise sequencing at least some of the products, thereby obtaining the sequence of a DNA fragment in the initial sample. As would be apparent, in these embodiments, the primers used for amplification step may contain 5′ tails containing sequences that are compatible with use in the sequencing platform being used for sequencing or, alternatively, the amplification products themselves may be amplified using primers that contain 5′ tails containing sequences that are compatible with use in the sequencing platform being used for sequencing.

In this embodiment, if the template after transposase insertion is long enough (e.g., >400 b or so) it should be possible to cyclize the template as duplex DNA; DNAs<400 bp may be under too great of torsional strain for efficient ligation. However, shorter fragments may be cyclized using CircLigase enzyme, following the protocol outlines in Lou at al. (PNAS 2013 110: 19872-19877). This method may require intact 3′ OH and 5′ phosphorylated ends, which may need to be created enzymatically by digestion or end polishing methods. One potential advantage of the cyclization method is that there could potentially be multiple unique molecular identifiers for each target. For example, if there are 1000 DNA fragments covering exon1 of the KRAS gene, the individual templates can be counted and distinguished by the following unique molecular signatures. First, most fragments will have different 5′ and 3′ ends. These ends will be ligated together, and can be resolved when the sequenced fragment is mapped to the genome. This is in contrast to typical tagmentation protocols, where the ends of the fragments are defined by transposase insertion, rather than by random shearing or fragmentation. Second, for a given target sequence, the transposase is likely to have different insertion sites in different DNA templates (this is similar to other tagmentation protocols). Finally, each transposase will have a molecular barcode (“NNNN . . . ”) which will uniquely identify each transposon. In this embodiment, three pieces of information can be used to identify and correct sequencing errors with very high confidence. The extra levels of barcoding may also be useful, or even required, for sequencing platforms which have a higher intrinsic error rate.

In any of the methods described above, the method may further comprise determining how many DBR sequences are associated with a particular sequence, thereby providing an estimate of the number of copies of that sequence in the initial sample. Such methods are described in Casbon (Nuc. Acids Res. 2011, 22 e81) among other publications.

In certain embodiments, the initial DNA being analyzed may be derived from a single source (e.g., a single organism, virus, tissue, cell, subject, etc.), whereas in other embodiments, the nucleic acid sample may be a pool of nucleic acids extracted from a plurality of sources (e.g., a pool of nucleic acids from a plurality of organisms, tissues, cells, subjects, etc.), where by “plurality” is meant two or more. As such, in certain embodiments, a transposon tagged sample may be combined with transposon tagged samples from other sources, e.g., 2 or more sources, 3 or more sources, 5 or more sources, 10 or more sources, 50 or more sources, 100 or more sources, 500 or more sources, 1000 or more sources, 5000 or more sources, up to and including about 10,000 or more sources, where the molecular barcode of the transposon allows the sequences from different sources to be distinguished after they are analyzed.

### Kits

Also provided by this disclosure are kits for practicing the subject method, as described above. In certain embodiments, the kit may comprise a plurality of barcoded transposons, a transposase, a terminal transferase, a forward primer that hybridizes to the bottom strand of the transposon, a reverse primer that hybridizes to the top strand of the transposon; and an oligo-dN primer. In other embodiments, the kit may comprise a plurality of barcoded transposons, a transposase, a forward primer that hybridizes to the bottom strand of the transposon and a reverse primer that hybridizes to the top strand of the transposon.

Either of the kits may additionally comprise suitable reaction reagents (e.g., buffers etc.) for performing the method. The various components of the kit may be present in separate containers or certain compatible components may be precombined into a single container, as desired. In addition to the reagents described above, a kit may contain any of the additional components used in the method described above, e.g., one or more enzymes and/or buffers, etc.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EMBODIMENTS

### Embodiment 1

A method comprising: (a) contacting a plurality of barcoded transposons and a transposase with a population of DNA fragments comprising DNA fragments of less than 1 kb in length, to produce transposon-tagged fragments that each comprise a barcoded transposon; (b) adding an oligo-dN tail to both ends of the transposon-tagged fragments using a terminal transferase; (c) amplifying, by PCR, a population of 5′ products each comprising a barcode using i. a reverse primer that hybridizes to the top strand of the transposon and ii. a primer that hybridizes to the oligo-dN tail; and (d) amplifying, by PCR, a population of 3′ products each comprising a barcode using i. a forward primer that hybridizes to the bottom strand of the transposon and ii. the primer that hybridizes to the oligo-dN tail.

### Embodiment 2

The method of embodiment 1, further comprising sequencing at least some of the 5′ products of step (c) and 3′ products of step (d) to obtain a plurality of 5′ sequence reads and a plurality of 3′ sequence reads, wherein the sequence reads each comprise i. the sequence of at least part of the sequence of a DNA fragment of (a) and ii. the sequence of a barcode.

### Embodiment 3

The method of embodiment 2, further comprising comparing the barcodes in at least some of the sequence reads to one another to obtain matches.

### Embodiment 4

The method of embodiment 3, further comprising assembling matched sequence reads to obtain the entire sequence of a DNA fragment of step (a).

### Embodiment 5

The method of any prior embodiment, further comprising determining how many barcode sequences are associated with a particular sequence, thereby providing an estimate of the number of copies of that sequence in the population of DNA fragments of step (a).

### Embodiment 6

The method of any prior embodiment, wherein the transposase of (a) is a Vibrio harveyi transposase, or a variant thereof.

### Embodiment 7

The method of any prior embodiment, wherein the transposase of (a) a Tn5 transposase, or a variant thereof.

### Embodiment 8

The method of any prior embodiment, wherein the population of DNA fragments is isolated from clinical sample.

### Embodiment 9

The method of embodiment 8, wherein the clinical sample is cell-free DNA extracted from a bodily fluid.

### Embodiment 10

The method of embodiment 9, wherein the bloodily fluid is blood.

### Embodiment 11

The method of embodiment 8, wherein the clinical sample is FFPE sample.

### Embodiment 12

A kit comprising: a plurality of barcoded transposons; a transposase; a terminal transferase; a forward primer that hybridizes to the bottom strand of the transposon; a reverse primer that hybridizes to the top strand of the transposon; and an oligo-dN primer.

### Embodiment 13

A method comprising: (a) contacting a plurality of barcoded transposons and a transposase with a population of DNA fragments comprising DNA fragments of less than 1 kb in length, to produce transposon-tagged fragments that each comprise a barcoded transposon; (b) circularlizing the transposon-tagged fragments; and (c) amplifying, by inverse PCR, a population of products each comprising a barcode and the sequence of a DNA fragment using i. a reverse primer that hybridizes to the top strand of the transposon and ii. a forward primer that hybridizes to the bottom strand of the transposon.

### Embodiment 14

The method of embodiment 13, further comprising sequencing at least some of the products of step (c), thereby obtaining the sequence of a DNA fragment of (a).

### Embodiment 15

The method of embodiment 14, wherein the method further comprises determining how many DBR sequences are associated with a particular sequence, thereby providing an estimate of the number of copies of that sequence in the population of DNA fragments of step (a).

### Embodiment 16

The method of any of embodiments 13-15, wherein the method comprises polishing the ends of the transposon-tagged fragments between steps (a) and (b).

### Embodiment 17

The method of any of embodiments 13-16, wherein the transposase of (a) is a Vibrio harveyi transposase, or a variant thereof.

### Embodiment 18

The method of any of embodiments 13-17, wherein the transposase of (a) is a Tn5 transposase, or a variant thereof.

### Embodiment 19

The method of any of embodiments 13-18, wherein the population of DNA fragments is isolated from clinical sample.

### Embodiment 20

The method of embodiment 19, wherein the clinical sample is cell-free DNA extracted from a bodily fluid.

### Embodiment 21

The method of embodiment 20, wherein the bloodily fluid is blood.

### Embodiment 22

The method of embodiment 19, wherein the clinical sample is FFPE sample.

### Embodiment 23

A kit comprising: a plurality of barcoded transposons; a transposase; a forward primer that hybridizes to the bottom strand of the transposon; a reverse primer that hybridizes to the top strand of the transposon.

1. A method comprising:
(a) contacting a plurality of barcoded transposons and a transposase with a population of DNA fragments comprising DNA fragments of less than 1 kb in length, to produce transposon-tagged fragments that each comprise a barcoded transposon;
(b) adding an oligo-dN tail to both ends of the transposon-tagged fragments using a terminal transferase;
(c) amplifying, by PCR, a population of 5′ products each comprising a barcode using i. a reverse primer that hybridizes to the top strand of the transposon and ii. a primer that hybridizes to the oligo-dN tail; and
(d) amplifying, by PCR, a population of 3′ products each comprising a barcode using i. a forward primer that hybridizes to the bottom strand of the transposon and ii. the primer that hybridizes to the oligo-dN tail.

2. The method of claim 1, further comprising sequencing at least some of the 5′ products of step (c) and 3′ products of step (d) to obtain a plurality of 5′ sequence reads and a plurality of 3′ sequence reads, wherein the sequence reads each comprise i. the sequence of at least part of the sequence of a DNA fragment of (a) and ii. the sequence of a barcode.

3. The method of claim 2, further comprising comparing the barcodes in at least some of the sequence reads to one another to obtain matches.

4. The method of claim 3, further comprising assembling matched sequence reads to obtain the entire sequence of a DNA fragment of step (a).

5. The method of claim 1, further comprising determining how many barcode sequences are associated with a particular sequence, thereby providing an estimate of the number of copies of that sequence in the population of DNA fragments of step (a).

6. The method of claim 1, wherein the transposase of (a) is a Vibrio harveyi transposase, or a variant thereof or a Tn5 transposase, or a variant thereof.

7. The method of claim 1, wherein the population of DNA fragments is isolated from clinical sample.

8. The method of claim 7, wherein the population of DNA fragments is cell-free DNA extracted from a bodily fluid.

9. The method of claim 8, wherein the bodily fluid is blood.

10. The method of claim 7, wherein the clinical sample is FFPE sample.

11. A kit comprising:
a plurality of barcoded transposons;
a transposase;
a terminal transferase;
a forward primer that hybridizes to the bottom strand of the transposon
a reverse primer that hybridizes to the top strand of the transposon; and
an oligo-dN primer.

12. A method comprising:
(a) contacting a plurality of barcoded transposons and a transposase with a population of DNA fragments comprising DNA fragments of less than 1 kb in length, to produce transposon-tagged fragments that each comprise a barcoded transposon;
(b) circularizing the transposon-tagged fragments; and
(c) amplifying, by inverse PCR, a population of products each comprising a barcode and the sequence of a DNA fragment using i. a reverse primer that hybridizes to the top strand of the transposon and ii. a forward primer that hybridizes to the bottom strand of the transposon.

13. The method of claim 12, further comprising sequencing at least some of the products of step (c), thereby obtaining the sequence of a DNA fragment of (a).

14. The method of claim 13, wherein the method further comprises determining how many DBR sequences are associated with a particular sequence, thereby providing an estimate of the number of copies of that sequence in the population of DNA fragments of step (a).

15. The method of claim 12, wherein the method comprises polishing the ends of the transposon-tagged fragments between steps (a) and (b).

16. The method of claim 12, wherein the transposase of (a) is a Vibrio harveyi transposase, or a variant thereof or a Tn5 transposase, or a variant thereof.

17. The method of claim 12, wherein the population of DNA fragments is isolated from clinical sample.

18. The method of claim 17, wherein the population of DNA fragments is cell-free DNA extracted from blood.

19. The method of claim 17, wherein the clinical sample is FFPE sample.

20. A kit comprising:
a plurality of barcoded transposons;
a transposase;
a forward primer that hybridizes to the bottom strand of the transposon; and
a reverse primer that hybridizes to the top strand of the transposon.
